(19) United States
(12) Patent Application Publication
(10) Pub. No.: US 2003/0013145 A1
(43) Pub. Date: Jan. 16, 2003

(54) Personal breast cancer indicator

(76) Inventor: Ted Titmas, Mission Viejo, CA (US)

Correspondence Address: OPPENHEIMER WOLFF & DONNELLY LLP, 840 NEWPORT CENTER DRIVE, SUITE 700, NEWPORT BEACH, CA, 92660, US

(21) Appl. No.: 10/203,696

(22) PCT Filed: Feb. 26, 2001
(86) PCT No.: PCT/US01/06120

(51) Int. Cl. G01N 33/574; C12M 1/34

(52) U.S. Cl. 435/7.23; 435/287.2

(57) ABSTRACT

A personal breast cancer testing kit utilizing breast cancer marker-sensitive pads which are stored in a package that permits the storage of the breast cancer marker-sensitive pads within its interior without exposing the breast cancer marker-sensitive pads to ultraviolet light, moisture or air. The kit comprises at least one pad being chemically sensitive to a predetermined breast cancer marker. The breast cancer marker-sensitive pads are stored in a package that is preferably formed from high grade surgical aluminum foil and paper and that is free from air.

### FIELD OF THE INVENTION

The present invention relates in general to personal testing kits for determining whether a woman has breast cancer. The kit tests for the presence of elevated levels of a marker found in the woman's saliva, which occurs in increased levels when the woman has developed breast cancer. The present invention relates more particularly to the packaging for storing the breast cancer marker-sensitive pads of a personal breast cancer testing kit and to the method for testing the woman's saliva to determine whether she has breast cancer. The invention is also applicable to the storage of the breast cancer marker-sensitive pads which must be protected from any contact with ultraviolet light, moisture, or air.

### BACKGROUND OF THE INVENTION

At the present time, there are no known tests for early detection of breast cancer that can be used by a woman in the privacy of her own home. A woman must rely on a breast exam that is done simply by her own hands to detect the presence of a lump. All too often, however, by the time a woman detects a lump by this method, the cancer has grown within the breast and may have spread into other parts of her body. This method is thus not acceptable for early detection of breast cancer.

There thus exists the need for a personal breast cancer test that can detect breast cancer in its earliest stages—before it has taken hold within the breast, and certainly before it spreads to any other parts of the woman's body. The test would be even more practical if it could be performed at home by the woman, without the use of expensive equipment and without the need for a doctor's assistance. The kit should also be self-contained, that is, all the necessary items, including instructions and any charts or graphs, should be included within or on the packaging. The test kit should also be easy to administer—easy enough so that any one can use the test kit without any specialized training.

It has recently been determined that, when a woman contracts breast cancer, there are one or more chemical markers that are placed into the woman's saliva. Accordingly, a testing kit that is able to detect one or more of these markers by testing the user's saliva may be capable of meeting these needs. One example of the testing of a person's saliva is the determination of a person's blood alcohol level. A person's saliva contains the same concentration of alcohol as in that person's blood. Test kits have been developed for detecting the level of a person's blood alcohol in that person's saliva. These test kits utilize alcohol-sensitive pads that are formed from paper saturated with alcohol oxidase enzyme, alcohol peroxidase enzyme, dyes and a buffer. When these alcohol-sensitive pads are saturated with a person's saliva, they will change color according to the level of alcohol in that saliva. Such a personal blood alcohol testing kit is disclosed in U.S. Pat. No. 5,563,073.

Accordingly, there exists a need for a personal testing kit to detect the presence of the marker for breast cancer in a woman.

### SUMMARY OF THE INVENTION

In accordance with the present invention, a personal breast cancer testing kit that is capable of determining whether a woman has contracted breast cancer is provided. The test kit detects certain markers that are present in the woman's saliva when she contracts breast cancer.

The test kit is comprised of the packaging and the pad or pads that are stored within the packaging. The pads are chemically sensitive to one or more of the markers for breast cancer found in the saliva of a woman who has contracted breast cancer and are preferably impregnated with at least one enzyme that is sensitive to the particular marker that it is searching for, at least one dye, and at least one buffer. The pads change color when it is contacted with the user's saliva, but only if the user's saliva contains the particular breast cancer marker for which the pad is intended to test. This change in color indicates that the woman's saliva contains this particular breast cancer marker, and thus the test will reveal by this change in color that the woman has contracted breast cancer.

The test kit of the present invention provides the benefit that it is capable of detecting breast cancer at an early stage in its development—often well-before a woman could ever detect a lump in her breast, which may be too late. This is because the enzyme pads of the present invention are capable of detecting the marker for breast cancer in the woman's saliva. This marker may be present in the woman's saliva, even before a lump appears in the breast. Thus, if the woman were to aggressively test her saliva for the breast cancer marker, even before she were to detect a lump, she may be able to detect the presence of the breast cancer well-before she ever would have by testing for a lump. Of course, such early detection provides the benefit of allowing the woman to get breast cancer treatment as soon as possible before the spread of the cancer.

Each breast cancer marker-sensitive pad comprises a piece of paper saturated with one or more enzymes that are sensitive to the breast cancer marker that is being tested. The paper is also saturated with dyes and a buffer. The breast cancer marker-sensitive pads are preferably cut into small pieces less than one-half square inch in size. The breast cancer marker-sensitive pads are attached to the end of a stick so that the user may place the breast cancer marker-sensitive pad within her mouth to saturate the pad with saliva or may dip the breast cancer marker-sensitive pad into saliva that has been collected in a cup.

The breast cancer marker-sensitive pads are stored in a package. The package includes a first side wall comprised of a plurality of layers coupled together with at least one of the layers being formed from aluminum foil and a second side wall also comprised of a plurality of layers coupled together with at least one of the layers being formed from aluminum foil. The aluminum foil of the first and second side walls of the package may be formed from high-grade surgical aluminum foil. Similarly, the aluminum foil may be coated with a polymer. In either case, the package is formed by coupling together the outer edge of the first side wall with the outer edge of the second side wall to form an interior for storing at least one breast cancer marker-sensitive pad. The package is intended to protect the breast cancer marker-sensitive pads from ultraviolet light, moisture, and air.

The instructions for use may be printed on the outside surface of one of the side walls of the packaging. Thus, by printing the instructions for use on the exterior of the package, a self-contained personal breast cancer testing kit is provided.

The user performs the personal breast cancer test after abstaining from eating or drinking for at least fifteen minutes prior to taking the test. The test is administered by saturating the breast cancer marker-sensitive pad or pads at the end of the stick with the user's saliva. Once the pad or pads are completely saturated, the user waits approximately two minutes for the breast cancer marker-sensitive pad or pads to change color. If any of the pads changes color, this indicates the presence of the breast cancer marker for which that particular pad is testing. If there is no change in color, then that breast cancer marker is not present. Thus, an easy-to-administer test is provided that is accurate and reproducible.

Other objects, features and advantages of the invention will become apparent from a consideration of the following detailed description and from the accompanying drawings.

### BRIEF DESCRIPTION OF THE DRAWINGS

FIG. 1 is a top plan view of the package for storing the breast cancer marker-sensitive pads of a personal breast cancer testing kit showing a plurality of the breast cancer marker-sensitive pads and sticks with which one embodiment of the present invention can be used;

FIG. 2 is a side cross-sectional view taken along the line 2-2 in FIG. 1 containing four sticks with pads; and

FIG. 3 is a top elevational view of the exterior of the package showing the instructions for use and color changes printed thereon with which one embodiment of the present invention can be used.

### DETAILED DESCRIPTION OF THE PREFERRED EMBODIMENTS

The present invention is embodied in a personal breast cancer testing kit for testing the presence of any one of a number of breast cancer markers in a user and includes the breast cancer marker-sensitive pads and the packaging for storing the breast cancer marker-sensitive pads.

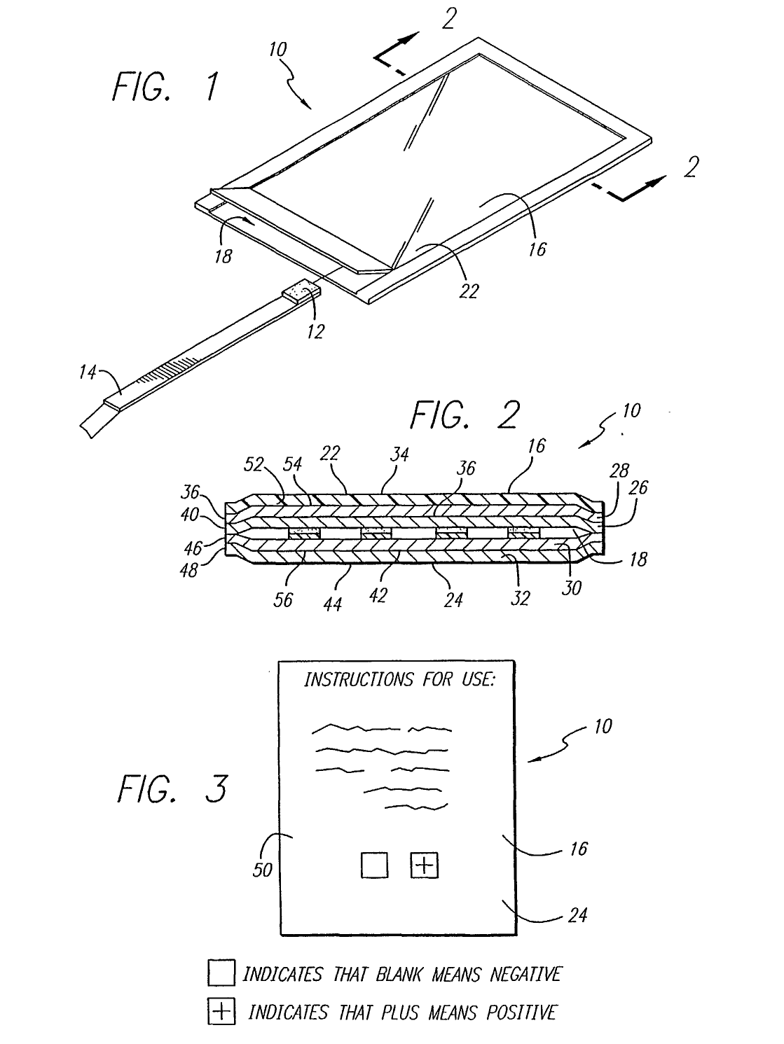

In the particular embodiment shown in the drawings and herein described, the personal breast cancer testing kit 10 (see FIGS. 1 and 2) comprises at least one breast cancer marker-sensitive pad 12 that is attached to a stick 14. The sticks 14 and pads 12 are stored within a package 16. The breast cancer marker-sensitive pads 12 may include one or more enzymes. These enzymes are sensitive to ultraviolet light, moisture, and air and will lose their activity if exposed to these elements for any substantial amount of time. Accordingly, these pads need to be stored in a package which prevents their exposure to these elements.

Each breast cancer marker-sensitive pad 12 is designed so that, when exposed to a person's saliva, the pads 12 are capable of detecting the presence of a predetermined breast cancer marker. If the breast cancer marker that the pad is designed to detect is in fact detected, buffers and dyes, that are also present on the breast cancer marker-sensitive pad 12, are activated so that they will cause a change in color of the pad 12. The user can then examine the change in color and determine whether they have developed breast cancer.

At least one breast cancer marker-sensitive pad 12 is provided within the interior 18 of the package 16. (FIGS. 1 and 2). The breast cancer marker-sensitive pads 12 are preferably formed from a piece of paper saturated with one or more enzymes, one or more dyes, and one or more buffers. Each breast cancer marker-sensitive pad is designed to be sensitive to a particular, specific breast cancer marker found in the saliva of a woman. Thus, this pad would be saturated with the enzyme or enzymes that are sensitive to one or more of the known breast cancer markers that will be present in a person's saliva when the woman has developed breast cancer and elevated levels of these markers are found in the person's saliva. In addition, this pad 12 should further include one or more dyes and buffers which will cause a change in color when the breast cancer marker enzyme or enzymes are activated by the breast cancer marker in the saliva. Any enzyme, dye, and buffer, or combination thereof, may be selected, so long as the enzyme, dye, and buffer combination are capable of detecting the presence of the marker for breast cancer the person's saliva, and, when such marker is detected, are capable of causing the breast cancer marker-sensitive pad to change color. Any enzyme may be chosen so long as it is capable of detecting the marker for elevated levels of a breast cancer marker in the user's saliva that indicates that the user has breast cancer. The breast cancer markers which may be detected by the breast cancer marker-sensitive pads in accordance with the present invention are any breast cancer marker which may be found in the woman's saliva as is known to those of ordinary skill in the art.

The fact that the test kit of the present invention is capable of detecting the presence of elevated levels of breast cancer markers makes the breast cancer test kit of the present invention capable of detecting whether the woman has developed breast cancer in the earliest stages of the disease. This is because the breast cancer marker that appears in the woman's saliva will appear while the disease is in its earliest stages—often well before the woman would be able to detect a lump in her breast.

The breast cancer marker-sensitive pads 12 are preferably mounted by any adhesive well-known in the art to one end of a thin plastic stick 14. (See FIGS. 1 and 2). Any type of plastic that can be formed into thin flexible sticks that is well-known in the art may be used to form stick 14. The stick 14 is preferably as wide as the breast cancer marker-sensitive pad 12 and long enough so that the user can place the breast cancer marker-sensitive pad within her mouth without placing the user's hand into the user's mouth. Additionally, the stick may be formed from any other substantially rigid and inert material, such as wood or plastic, if desired.

Each breast cancer marker-sensitive pad 12 is cut into small pieces and are rectangularly shaped, preferably being one quarter inch long by three-eighths inches wide. If desired, the pads 12 may be cut into any shape so long as the pad will fit onto the end portion of the stick 14 that can be easily inserted into the user's mouth and saturated with saliva. The end of the stick 14 that contains the pads may be wider than the remainder of the stick that does not contain the pads 12 to make it clear to the user which portion of the stick 14 should be saturated with the saliva.

The enzymes that are used on the pads 12 are sensitive to exposure to ultraviolet light, moisture, and air and will lose their activity if they are exposed to any of these elements for an appreciable amount of time. Thus, the package 16 of the present invention is particularly suited for storing the breast cancer marker-sensitive pads 12 of the testing kit 10 without exposing them to ultraviolet light, moisture, or air. The package 16 preferably comprises a first side wall 22 and a second side wall 24. Both the first and second side walls (22 and 24, respectively) preferably comprise an inner and outer layer, (26 and 28, respectively, for the first side wall and 30 and 32, respectively, for the second side wall). The inner layer 26 of the first side wall 22 and the inner layer 30 of the second side wall 24 is preferably formed from aluminum foil.

The aluminum foil used is preferably a high grade surgical aluminum foil. It has been found that the high grade surgical aluminum foil is preferred over other types of aluminum foils because this type of aluminum foil does not give off any oils that could damage the breast cancer marker-sensitive pads during prolonged storage times. Conventional aluminum foils that are typically used in packaging may give off the oils and residues that are imparted to the aluminum foil during manufacture when exposed to elevated temperatures, such as room temperature and the temperatures found when stored in a warehouse or on an automobile dashboard. For this reason, high grade surgical aluminum foil may be chosen instead. High grade surgical aluminum foil differs from conventional aluminum foil in that it is specially washed during manufacture to remove the impurities normally found in conventional aluminum foil. Thus, the term high grade surgical aluminum foil is used herein to describe aluminum foil that is in a condition wherein it is free from impurities such as oils and other foreign matter. The use of the high grade surgical aluminum foil therefore provides the distinct benefit of allowing the breast cancer marker-sensitive pads 12 of the present invention to be stored within a package 16, without exposing the pads 12 to ultraviolet light, moisture, air, or any other impurities. Further, the packaging provides the additional benefit that the pads 12 do not have to be stored under refrigerated conditions, but, rather can be stored at room temperature, or even in a warehouse or on the dashboard of a car.

In the alternative, the inner layers 26, 30 of the first and second side walls 22 and 24 of the packaging 16 may comprise conventional aluminum foil which has been coated on at least one surface with a polymer. If the surface of the aluminum foil having the polymer is utilized as the inner layer of the side walls that contacts the breast cancer marker-sensitive pads, then the polymer coating will prevent the oils of the conventional aluminum foil from coming into contact with the breast cancer marker-sensitive pads that are located within the package. Any polymer and any means for coating the conventional aluminum foil known in the art may be used, so long as the foil/polymer combination are capable of providing a packaging which prevents the breast cancer marker-sensitive pads from coming into contact with ultraviolet light, moisture, or air.

The outer layer 28 of the first side wall 22 and the outer layer 32 of the second side wall 24 are preferably formed from paper. By providing a first and second side wall, 22 and 24, that are formed from paper and aluminum, the first and second side walls of the package 28, 32, are easily opened by tearing by the user. Although paper is preferred as the outer layer of the first and second side walls, 22, 24, any material may be used that will provide a carrier for the aluminum foil that comprises the inner layer 26, 30 of the side walls 22, 24 and provide the benefit of preventing exposure of the breast cancer marker-sensitive pads contained within the packaging to ultraviolet light, moisture, and any other impurities.

As shown in FIG. 2, the first side wall 22 is formed by coupling together the surface 34 of the layer 28 of the first side wall 22 with the surface 36 of the inner layer 26 of the first side wall 22. When coupled together, the outer edge 38 of the outer layer 28 is preferably contiguous with the outer edge 40 of the inner layer 26. Any type of adhesive material that is well-known in the art for coupling aluminum foil to paper may be used for coupling together these two layers. The second side wall 24 is formed by coupling together the surface 42 of the inner layer 30 of the second side wall 24 with the surface 44 of the outer layer 32 of the second side wall 24. When coupled together, the outer edge 46 of inner layer 30 is preferably contiguous with the outer edge 48 of the outer layer 32. Again, any adhesive well-known in the art may be used for coupling these layers together.

The package 16 for storing the breast cancer marker-sensitive pads of the personal breast cancer testing kit 10 of the present invention is then preferably formed by coupling together the outer edge 40 of the inner layer 26 of the first side wall 22 with the outer edge 46 of the inner layer 30 of the second side wall 24 to form an interior 18 for storing at least one breast cancer marker-sensitive pad 12. (See FIG. 2). The inner layer 26 of the first side wall 22 and the inner layer 30 of the second side wall 24, which are both preferably formed from high grade surgical aluminum, are facing each other and form the inside lining of the interior 18 of the package 16.

When the first side wall 22 and the second side wall 24 are coupled together, the breast cancer marker-sensitive pads 12, which are attached to sticks 14, are placed between the two side walls in a vacuum environment so that the breast cancer marker-sensitive pads do not contact any air when they are placed into the package 16 or when they are stored in the interior 18 of the package 16. Thus, the breast cancer marker-sensitive pads 12 of the testing kit 10 are maintained within an environment in which their only contact is with the inner layer 26 of the first side wall 22 and the inner layer 30 of the second side wall 24, both preferably formed from the high grade surgical aluminum foil. The exterior of the package 16 that is exposed to ultraviolet light, moisture and air, are the outer layer 28 of the first side wall 22 and the outer layer 30 of the second side wall 32, which are formed from paper. The package 16, having the high grade surgical aluminum foil interior (or polymer coated aluminum interior) and paper exterior is able to store the breast cancer marker-sensitive pads 12 of the testing kit in its interior 18 without exposing the breast cancer marker-sensitive pads 12 to any ultraviolet light, moisture or air.

The instructions for administering the breast cancer test may also be printed on the exterior 50 of the package 16 along with the location of the particular pads, as described above. By printing the instructions and the location of the pads, a self-contained personal breast cancer test is provided, that is, the entire test is contained within and on the packaging.

The breast cancer marker-sensitive pads 12 are designed to change color if the breast cancer marker is detected in the user's saliva. In another alternative, the pads will change such that a symbol, such as a plus sign, will appear if the breast cancer marker is detected. Thus, if the breast cancer marker is detected, the pad 12 will change color or a symbol will appear and the test will be positive for breast cancer. If no breast cancer marker is detected, there will be no change in color or no symbol will appear and the test for breast cancer is negative. The user would therefore be capable of easily and quickly determining the results of the test.

The user preferably performs the breast cancer test as follows. The user opens the package 16 by tearing open the first and second side walls, 22 and 24, respectively, to reveal the breast cancer marker-sensitive pads 12 and sticks 14 within the interior 18. The user then preferably saturates the breast cancer marker-sensitive pads 12 with the user's saliva, preferably by either placing the breast cancer marker-sensitive pad end of the stick 14 into the mouth and saturating the breast cancer marker-sensitive pad 12 with saliva. The user may also place their saliva in a cup and saturate the breast cancer marker-sensitive pad 12 in the saliva. The user then preferably waits approximately two minutes for the breast cancer marker-sensitive pad 12 to become activated and change color, if the presence of the breast cancer marker being tested is detected. If no color change is detected or no symbol appears, then the test is negative for breast cancer. If the pad 12 changes color, then the user has breast cancer. Thus, as can be seen, the test is easy to administer, is self-contained, and requires no other outside parts other than the user's saliva to perform.

In a further preferred embodiment of the present invention, the first and second side walls 22 and 24 respectively, are rectangularly shaped. It is preferable that the package containing the personal breast cancer testing kit be portable so that it can be discreetly carried in a wallet, pocket or purse. Thus, one size for making the first and second side walls 22, 24 is a size approximating that of a business card, which are commonly held within a wallet. This size is preferably rectangular, being three and a half inches long by two inches wide.

In another embodiment of the present invention, the first side wall 22 further comprises a plastic covering layer 52 coupled together with the upper surface 54 of the outer layer 28 of the first side wall 22. (See FIG. 2). The plastic covering layer 52 may also be coupled to the lower surface 56 of the outer layer 32 of the second side wall 24 (Not shown in FIG. 2). The plastic covering layer 52 is substantially rigid to protect the breast cancer marker-sensitive pads 12 and sticks 14 contained within the package 16 from any bending that may occur while the package is stored within a wallet, pocket or purse of the user. The-plastic covering layer is preferably formed from any plastic that can be formed into thin, substantially rigid sheets that can be imprinted with color printing.

While a particular form of the invention has been illustrated and described, it will be apparent that various modifications can be made without departing from the scope of the invention. For instance, the size and shape of the first and second side walls 22, 24 may be chosen as preferred by the manufacturer in any size and shape so long as the sticks 14 and the breast cancer marker-sensitive pads 12 are contained within the interior of the package. Accordingly, it is not intended that the invention be limited by the specific embodiment disclosed in the drawings and described in detail hereinabove.

I claim:

1. A personal breast cancer testing kit, comprising:
at least one pad being chemically sensitive to a predetermined breast cancer marker; and
a package, the pads being stored within the package.

2. The personal breast cancer testing kit of claim 1 further including at least one stick having an end wherein the pad is attached to the end of the stick and wherein the stick is stored within the package.

3. The personal breast cancer testing kit of claim 1 wherein the pad is formed from paper and is impregnated with at least one enzyme that is sensitive to the predetermined marker for breast cancer, at least one dye and at least one buffer, wherein when the breast cancer marker-sensitive enzyme comes in contact with the user's saliva, the dye changes color only if the predetermined breast cancer marker is present in elevated amounts in the system of the user.

4. The personal breast cancer testing list of claim 3, wherein the dye is formed into the shape of a symbol on the pad.

5. The personal breast cancer testing kit of claim 4, wherein the symbol is a plus sign.

6. The personal breast cancer testing kit of claim 1 wherein the package has an exterior having the instructions for use of the personal breast cancer testing kit printed thereon.

7. The personal breast cancer testing kit of claim 1 wherein the package has an exterior surface and an interior surface and wherein the interior surface comprises aluminum foil.

8. The personal breast cancer testing kit of claim 7, wherein the aluminum foil is high-grade surgical aluminum foil.

9. The personal breast cancer testing kit of claim 8, wherein the high grade surgical aluminum foil is coated with a polymer.

10. A personal breast cancer testing kit, comprising:
at least one pad being chemically sensitive to a predetermined breast cancer marker; and
a package having an interior being formed from high grade surgical aluminum foil, the pads being stored within the package.

11. The personal breast cancer testing kit of claim 10 further including at least one stick having an end wherein the pad is attached to the end of the stick and wherein the stick is stored within the package.

12. The personal breast cancer testing kit of claim 10 wherein the exterior of the package comprises paper.

13. The personal breast cancer testing kit of claim 10 wherein the pad is formed from paper and is impregnated with at least one enzyme that is sensitive to the predetermined breast cancer marker, at least one dye and at least one buffer, wherein when the breast cancer marker-sensitive enzyme comes in contact with the user's saliva, the dye changes color only if the predetermined breast cancer marker is present in elevated amounts in the system of the user.

14. The personal breast cancer testing list of claim 13, wherein the dye is formed into the shape of a symbol on the pad.

15. The personal breast cancer testing kit of claim 14, wherein the symbol is a plus sign.

16. The personal breast cancer testing kit of claim 10 wherein the package has an exterior having the instructions for use of the personal breast cancer testing kit printed thereon.

17. The personal breast cancer testing kit of claim 10 wherein the package has an enclosed interior, the enclosed interior containing substantially no air.

18. A personal breast cancer testing kit, comprising:
at least one pad being chemically sensitive to the predetermined breast cancer marker, the pad being formed from paper and being impregnated with at least one breast cancer marker-sensitive enzyme, at least one dye and at least one buffer, wherein when the breast cancer marker-sensitive enzyme comes in contact with the user's saliva, the dye changes color; and
a package for storing the pad, the package being formed from paper and high grade surgical aluminum foil.

19. The personal breast cancer testing list of claim 18, wherein the dye is formed into the shape of a symbol on the pad.

20. The personal breast cancer testing kit of claim 19, wherein the symbol is a plus sign.

21. The personal breast cancer testing kit of claim 18 wherein the package includes:
a first side wall having an outer edge and being comprised of a plurality of layers coupled together, at least one of the layers being formed from aluminum foil; and
a second side wall having an outer edge and being comprised of a plurality of layers coupled together, at least one layer being formed from aluminum foil,
wherein the outer edge of the first side wall is coupled together with the outer edge of the second side wall to form an interior for storing at least one breast cancer marker-sensitive pad.

22. The personal breast cancer testing kit of claim 21 further including at least one stick having an end wherein the breast cancer marker-sensitive pad is attached to the end of the stick and wherein the stick is stored within the interior of the package.

23. The personal breast cancer testing kit of claim 21 wherein one of the layers of the first side wall is a substantially rigid plastic layer.

24. The personal breast cancer testing kit of claim 21 wherein one of the layers of the first side wall is printed with the instructions for use of the breast cancer testing kit.

25. The personal breast cancer testing kit of claim 21 wherein at least one of the layers of the first side wall and at least one of the layers of the second side wall are formed from paper.

26. The personal breast cancer testing kit of claim 21 wherein the aluminum foil layer of the first side wall and the aluminum foil layer of the second side wall comprises a high grade surgical aluminum foil.

27. The personal breast cancer testing kit of claim 26 wherein the high grade surgical aluminum foil layer of the first side wall and the high grade surgical aluminum foil layer of the second side wall is coated with a polymer.

28. The personal breast cancer testing kit of claim 21 wherein the interior of the package contains substantially no air.

29. A method for determining whether a woman has breast cancer, comprising:
opening a package comprising aluminum foil and containing at least one pad being chemically sensitive to a predetermined breast cancer marker;
saturating one of the pads with the user's saliva; and
waiting approximately two minutes to determine whether the pad has detected the presence of the breast cancer marker by changing its color.

30. The method of claim 23 wherein the package comprises a high grade surgical aluminum foil.

31. The method of claim 23 wherein the high grade surgical aluminum foil of the package is coated with a polymer.
